(19) United States
(12) Patent Application Publication
(10) Pub. No.: US 2011/0137336 A1
(43) Pub. Date: Jun. 9, 2011

(54) SKIN PROTECTING TOURNIQUET APPARATUS

(76) Inventors: Paula Holcomb, Poulsbo, WA (US); Michael Ward Holcomb, Poulsbo, WA (US)

(21) Appl. No.: 12/960,399

(22) Filed: Dec. 3, 2010

Related U.S. Application Data
(60) Provisional application No. 61/266,467, filed on Dec. 3, 2009.

(51) Int. Cl. A61B 17/00

(52) U.S. Cl. 606/203

(57) ABSTRACT

An embodiment is a tourniquet including a constricting band with an angled constriction tightening point to substantially prevent skin from being caught in the constriction action. The angled constriction tightening point may further enable the tourniquet to substantially securely couple to web gear, for example to pouch attachment ladder system (PALS) webbing of modular lightweight load-carrying equipment (MOLLE) systems.

### CROSS-REFERENCE TO RELATED APPLICATION

This application is a nonprovisional application claiming priority to co-pending provisional patent application Ser. No. 61/266,467, filed Dec. 3, 2009, which is incorporated herein by reference.

### FIELD OF THE INVENTION

The present invention relates to tourniquets that integrate with tactical equipment and with teeth and locking mechanisms designed to protect the skin of the patient upon whom the tourniquet is applied.

### BACKGROUND

A tourniquet is designed for use in austere environments to mitigate patients massive bleeding from a patient's limb and to preferably amputation of the limb.

Existing tourniquets do not satisfy combat lifesaver needs. Existing tourniquets used for combat environments take a long time to apply, and are bulky when attached to personal equipment. Other tourniquet techniques used in combat environments that are not sufficient include using a cravat worn around the neck as a scarf until needed, tourniquets requiring sliding a cloth loop up over a limb and injury site and narrow gauge zip ties.

Specifically, zip ties and cable ties are known in the art to be useable on appendages. However, existing zip and cable ties can cause additional tissue damage due to their narrow width. Additionally they can pinch the skin when rapidly applied to a patient. This not only can give the patient additional minor injuries, but the trapped skin folds may also prevent the tourniquet from being tightened enough to be effective. Cloth loop tourniquets require additional time to apply as they need to either be fed through a buckle locking system, or slide up around the limb. The latter is the most ineffective as it requires the tourniquet to slide over the injury site, and with partial amputations and open fractures this is time consuming and presents extra risk to the patient.

Further, cravats are of limited use as they require constant pressure while applying and tying, and are bulky when stored. Additionally cravats can cause the patient minor skin injuries, in the form of a friction burn, as a cravat is applied. Finally all of the above methods do not include any feature or provision to record the time and date the tourniquet was applied, especially if the first responder is wearing gloves and/or is in a high stress situation and has reduced fine motor skills.

The following represents a list of known related art:

| | Date of | |
|---|---|---|
| Reference: | Issue/Publication: | Issued to: |
| U.S. Pat. No. 4,384,583 | May 24, 1983 | Speelman et al. |
| U.S. Pat. No. 4,125,115 | Nov. 114, 1978 | Mayo et al. |
| U.S. Pat. No. 4,911,162 | Mar. 27, 1990 | Wolff |
| U.S. Pat. No. 2,936,759 | May 17, 1960 | Yuhas |

The teachings of each of the above-listed citations (which does not itself incorporate essential material by reference) are herein incorporated by reference. None of the above inventions and patents, taken either singularly or in combination, is seen to describe the instant invention as claimed.

U.S. Pat. No. 4,384,583 to Speelman et al. for a “Tourniquet” discloses a tourniquet constructed from a length of elastic material. The ends of the elastic material are made non-elastic, and complementary coupling components are attached thereto. The elastic material is provided with a thickening for stress absorption and control.

U.S. Pat. No. 4,125,115 to Mayo et al. for a “Tourniquet” discloses an improved tourniquet. It has a semi-elastic belt fixed at one end to a tongue. The tongue is releasably engageable in a buckle. The other end of the belt passes through belt adjusting means to form a tourniquet loop. The belt adjusting means form part of the buckle. The length of the loop is adjustable as required. Slow and quick tourniquet release means are provided at the belt adjustment means and tongue engagement means, respectively.

U.S. Pat. No. 4,911,162 to Wolff for a “Tourniquet” discloses a tourniquet for restricting the flow of blood to and from a b of an individual when assembled thereon, the tourniquet having a flexible and resilient elongated member and a tourniquet securing member fixedly secured to one end of the flexible and resilient elongated member and having an aperture therethrough to receive a second end of the elongated member once the elongated member is wrapped about the limb of an individual so that the second end of the elongated member can be pulled against the inner peripheral surface of the aperture causing the tourniquet securing member to bear against the individual's limb and the tightening of the elongated member about the limb so as to enlarge and expose the veins or veinlets of the limb. The tourniquet securing member includes a cavity at one end to receive the elongated member wherein the cavity has gripping teeth therein to fixedly grip the flexible and resilient elongated member.

U.S. Pat. No. 2,936,759 to Yuhas for a “Tourniquet” discloses a compression devices and more particularly, a tourniquet readily applied in cases of accident to the largest or smallest members of the body to restrict the flow of blood and arrest bleeding

### SUMMARY AND ADVANTAGES

A tourniquet for protecting skin of a patient is provided. An embodiment is a tourniquet including a tail with a tip and teeth, a hood connected to the tail distal from the tip, an angled skin protecting neck extending from the hood, and a head with a mouth through which the tip and tail insert and pass attached to the neck. Another embodiment is a tourniquet integrally formed to include a tail having a tip and teeth, a hood on the end of the tail distal from the tip, an angled skin protecting neck extending from the hood, and a head a mouth through which the tip and tail insert and pass formed at the end of the neck. A further embodiment is a tourniquet including an arm constricting band with an angled constriction tightening point preventing skin from being caught in the constriction action.

The tourniquet of an embodiment present invention presents numerous advantages, including: (1) low cost to produce, (2) lighter than existing products, (3) faster to deploy and use than existing products, (4) reduced bulk when used on personal equipment such as flak jackets, (5) its stiffness allows it to slide under limbs without lifting them, (6) can be used in any climate and place, (7) wider tail portion may cause less pinch or other damage to underlying skin or tissue when the tourniquet is applied, (8) width is compatible with the personal equipment attachment system used by the current generation of military equipment, (9) the length may allow the tourniquet to be used on legs as well as arms, yet still attach to personal equipment such as a flak jacket, (10) if the patient has a large build, two or more skin protecting tourniquet apparatuses may be linked together, (11) the hood provides space for the first responder to write the date and time on the skin protecting tourniquet apparatus, (12) the neck lets the first responder tuck the head under a flak jacket strap to create a tension lock to hold the tourniquet substantially securely, (13) the angle of the head relative to the rest of the skin protecting tourniquet apparatus may prevent skin pinch when the tourniquet is rapidly applied.

Additional advantages of the invention will be set forth in part in the description which follows, and in part will be obvious from the description, or may be learned by practice of the invention. The advantages of the invention may be realized and attained by means of the instrumentalities and combinations particularly pointed out in the appended claims. Further benefits and advantages of the embodiments of the invention will become apparent from consideration of the following detailed description given with reference to the accompanying drawings, which specify and show preferred embodiments of the present invention.

### BRIEF DESCRIPTION OF THE DRAWINGS

The accompanying drawings, which are incorporated into and constitute a part of this specification, illustrate one or more embodiments of the present invention and, together with the detailed description, serve to explain the principles and implementations of the invention.

FIG. 1 shows a skin protecting tourniquet apparatus top down view.

FIG. 2 shows a side view of a skin protecting tourniquet apparatus.

FIG. 3 shows a portion of a skin protecting tourniquet apparatus.

FIG. 4 shows a skin protecting tourniquet apparatus side view with the tail through the head.

FIG. 5 shows a skin protecting tourniquet apparatus in use.

FIG. 6 shows a skin protecting tourniquet apparatus held on a flak jacket.

### REFERENCE NUMBERS USED IN DRAWINGS

Turning now descriptively to the drawings, in which similar reference characters denote similar elements throughout the several views, the figures illustrate a skin protecting tourniquet apparatus. With regard to the reference numerals used, the following numbering is used throughout the various drawing figures:
10 skin protecting tourniquet apparatus
12 tail
14 teeth
16 head
18 hood
20 neck
22 mouth
24 tip
A arm
F flak jacket

### DETAILED DESCRIPTION

Before beginning a detailed description of the subject invention, mention of the following is in order. When appropriate, like reference materials and characters are used to designate identical, corresponding, or similar components in differing figure drawings. The figure drawings associated with this disclosure typically are not drawn with dimensional accuracy to scale, i.e., such drawings have been drafted with a focus on clarity of viewing and understanding rather than dimensional accuracy.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application- and business-related constraints, and that these specific goals will vary from one implementation to another and from one developer to another. Moreover, it will be appreciated that such a development effort might be complex and time-consuming, but would nevertheless be a routine undertaking of engineering for those of ordinary skill in the art having the benefit of this disclosure.

A tourniquet to protect skin of a patient is provided. In an embodiment, the tourniquet may include a tail with a tip and teeth, a hood connected to the tail distal from the tip, an angled skin protecting neck extending from the hood, and a head with a mouth through which the tip and tail insert and pass attached to the neck. An alternate embodiment is a tourniquet integrally formed to include a tail having a tip and teeth, a hood on the end of the tail distal from the tip, an angled skin protecting neck extending from the hood, and a head with a mouth through which the tip and tail insert and pass formed at the end of the neck. A further embodiment is a tourniquet including an arm constricting band with an angled constriction tightening point preventing skin from being caught in the constriction action.

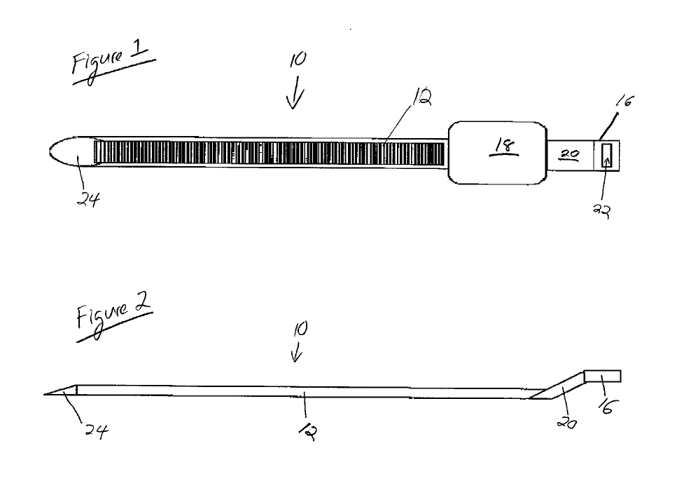

As shown in FIGS. 1-6, a skin protecting tourniquet apparatus 10 includes a tail 12 having teeth 14, a hood 18, an neck 20, and a head 16 with a mouth 22 through which the tail 12 tip 24 slides and which locks onto the teeth 14 on the tail 12 similar to a zip tie or cable tie. Preferably, the skin protecting tourniquet apparatus 10 is approximately 28 inches overall length. In an embodiment, the intersection of the hood 18 and the neck 20 may define a bend or angle.

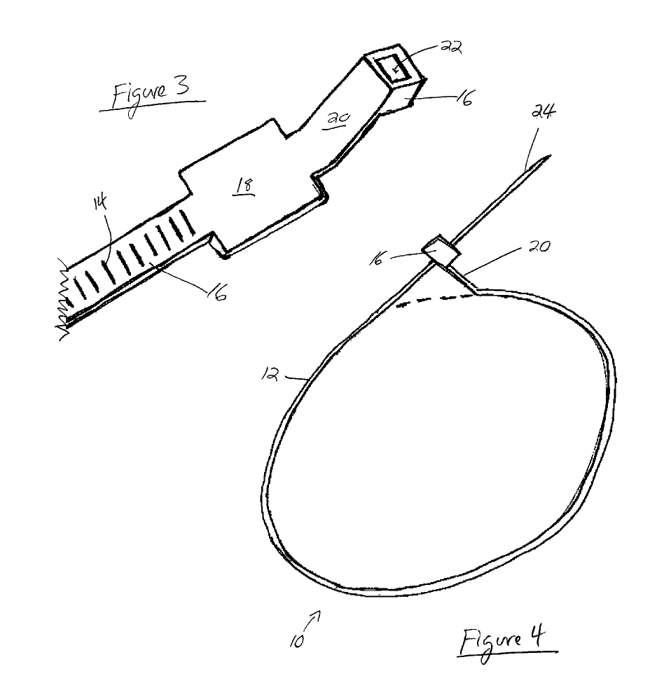

More specifically, FIGS. 2 and 3 illustrate that the tail 12 is connected to the hood 18, and the hood 18 in turn is connected to the neck 20. The hood 18 and neck 20 connected thereto may define a bend or angle, For example, the neck 20 may form an angle of approximately between 20 degrees and 40 degrees with the hood 18. In an embodiment, the angle may be approximately 30 degrees. At the end of the neck 20 is a head 16 including mouth 22 through which the tail 12 slides and substantially locks into place. The tail 12 of an embodiment may fit within the webbing and netting on flak jackets F, as shown in FIG. 6. For example, the tail 12 may be compatible with the PALS webbing (Pouch Attachment Ladder System) of current generation MOLLE (Modular Lightweight Load-carrying Equipment) load-bearing equipment and rucksacks utilized by military, law enforcement, and search and rescue personnel. More specifically, the PALS webbing includes rows of heavy-duty nylon stitched onto the vest or other piece of personal equipment in a grid including horizontal rows of 1.0 inch webbing, spaced 1.0 inch apart, and reattached to the backing at 1.5 inch intervals. The width of the tail 12 may be compatible with the loops formed in the horizontal rows of the PALS such that the skin protecting tourniquet apparatus 10 may be threaded through and removably attached to one or more horizontal rows. More specifically, the skin protecting tourniquet apparatus 10 may be removably stowed to the PALS in a vertical orientation and may be deployed akin to drawing a sword from a scabbard.

The tail 12 is preferably approximately 1.0 inch wide, by approximately 24 inches long, by approximately 0.1 inch thick. The first one inch of length (i.e., the end) of the tail 12 may be angled and/or tapered to facilitate and/or ease entry of the tail 12 into the mouth 22 of the head 16 to secure the skin protecting tourniquet apparatus 10. The tapered and/or angled end of the tail 12 may or may not include ridges and/or a texture to facilitate grasping the tail 12, but does not have locking teeth 14. The rest of the tail 12 includes locking teeth 14 formed therein to engage the mouth 22 and substantially lock the tail 12 in place.

In an embodiment, the mouth 22 may engage the locking teeth 14 of tail 12 unidirectionally. More specifically, the mouth 22 may substantially permit the insertion of the tail 12 while substantially preventing the removal of tail 12. Accordingly, the skin protecting tourniquet apparatus 10 may be lockably tightened to substantially maintain compression for example upstream from the patient's wound to mitigate hemorrhaging and resulting blood loss. In an embodiment, the skin protecting tourniquet apparatus 10 may not thereafter be loosened. For such an embodiment, removal of the skin protecting tourniquet apparatus 10 may require cutting the skin protecting tourniquet apparatus 10. Alternately, the head 16 and/or head 22 may include a release (not illustrated) to loosen, remove, and/or reuse the skin protecting tourniquet apparatus.

As noted, the skin protecting tourniquet apparatus 10 of an embodiment may substantially prevent skin pinch and/or other skin or tissue damage as the skin protecting tourniquet apparatus 10 is applied. For example, the approximate 1.0 inch width of the tail 12 is wider than a normal zip tie/cable tie and may create less tissue damage by increasing the surface area to which the pressure of a tightened skin protecting apparatus 10 is applied.

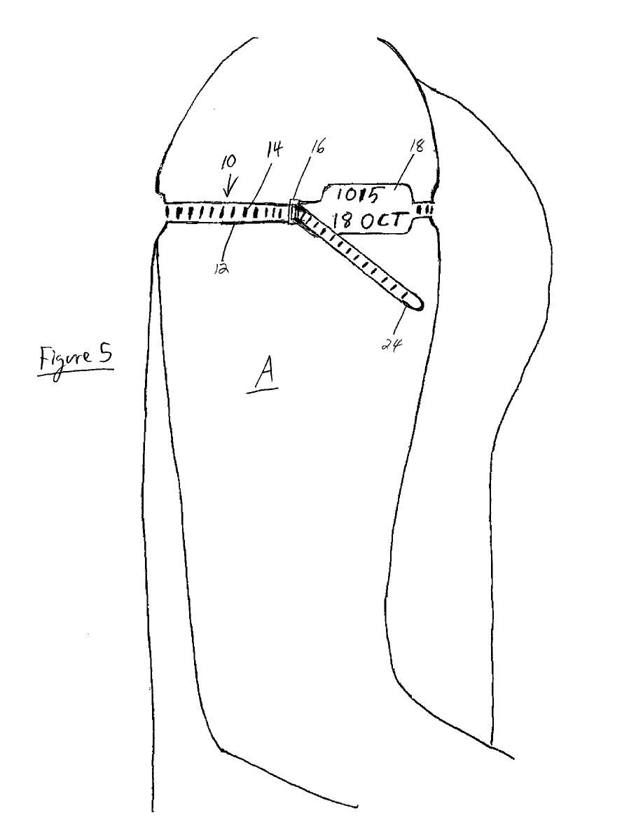

As illustrated by FIG. 4, the neck 20 may be approximately 1.0 inch long, by 1.0 inch wide, by 0.3 inch thick and may be angled relative to the tail 12 and/or hood 18. More specifically, the angle of the neck 20 may be molded into the skin protecting tourniquet apparatus 10 to yield an angle between the neck 20 and the tail 12 and/or hood 18 of approximately between 20 and 40 degrees. In an embodiment, the angle between the neck 20 and the tail 12 and/or hood 18 may be approximately 30 degrees. Said differently, the angle between the neck 20 and the tail 12 and/or hood 18 may be approximately 30 degrees plus or minus 10 degrees (30°±10°). In an embodiment, the when the skin protecting tourniquet apparatus 10 of an embodiment is deployed and applied to a patient, the angle between the neck 20 and the tail 12 and/or hood 18 may position and/or deflect the head 16 including mouth 22 away from the patient's skin. Accordingly, as the skin protecting tourniquet apparatus 10 is tightened, the patient's skin may not be pinched between the head 16 and the tail 12 and/or hood 18. Said differently, the bend or angle at the intersection of neck 20 and tail 12 and/or hood 18 may substantially prevent skin pinch.

As illustrated by FIG. 6, the bend in the skin protecting tourniquet apparatus 10 may further enable the skin protecting tourniquet apparatus 10 to more securely removably couple to and/or lock into the PALS webbing of, for example, MOLLE personal equipment including of tactical flak vest designs and the like. More specifically, the bend of an embodiment may function as a tension hook and/or tension lock as it fits under the weave of the PALS webbing or the like to f yore securely removably couple and/or substantially lock the skin protecting tourniquet apparatus 10 to the PALS webbing. Said differently, the bend or angle in the skin protecting tourniquet apparatus 10 may create a tension lock to substantially hold the skin protecting tourniquet apparatus 10 in place by first inserting, for example, the tail 12 through one or more PALS webbing loops (or the like) and then inserting the head 16 under another PALS webbing loop or strap. Inserting the head 16 under a separate PALS webbing loop or strap may in turn straighten the bend or angle and may substantially lock the skin protecting tourniquet 10 in place with tension and/or friction between the head 16 and the PALS webbing loop or strap.

The skin protecting tourniquet apparatus 10 of an embodiment may be provided with a vertical hanging storage device (not illustrated) with two elastic band loops into which multiple skin protecting tourniquet apparatuses 10 may fit. Further, the vertical hanging storage device may enable multiple skin protecting tourniquet apparatuses 10 to removably couple to personal equipment not MOLLE and/or compatible. In operation, a first responder may unhook the top of a skin protecting tourniquet apparatus 10 from the top elastic band loop, then slide the tail 12 up and out of the bottom elastic band loop. This will allow for a number of skin protecting tourniquet apparatuses 10 to be securely held, yet rapidly accessed. As with the MOLLE and/or PALS compatible embodiment, the angle between the neck 20 and the tail 12 of one or more skin protecting tourniquet apparatuses 10 may enable the skin protecting tourniquet apparatuses 10 to removably lock into place.

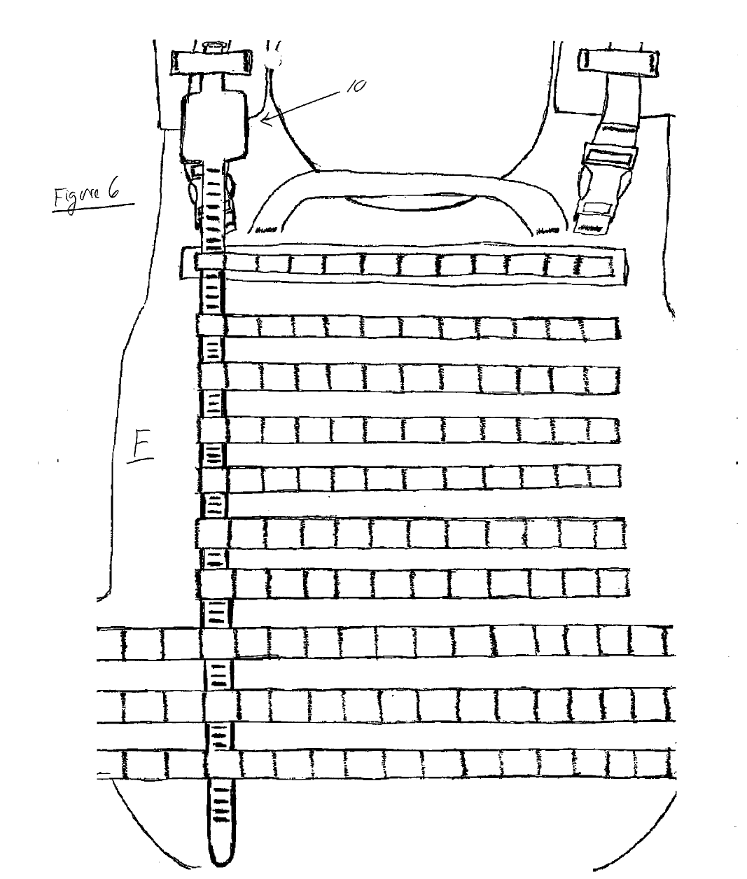

The hood 18 of skin protecting tourniquet apparatus 10 of an embodiment is preferably approximately 1.5 inches wide, by approximately 3.0 inches long, by approximately 0.1 inch thick. As illustrated by FIG. 5, the size and/or configuration of the hood 18 may provide sufficient space to allow a first responder to write the date and time of the skin protecting tourniquet apparatus 10 deployment and application and/or any other information relevant to the condition and treatment of the patient. The date and time of the deployment and application may be particularly important as the application of skin protecting tourniquet apparatus 10 for extended periods may be medically detrimental.

The skin protecting tourniquet apparatus 10 of an embodiment may comprise a material that is sufficiently stiff to be able to slide under limbs without lifting them, as when treating a patient lying on the ground, and sufficiently durable to allow them to be used in any climate and locale. Suitable materials include nylon, vinyl, polyvinyl compounds, plastics, and/or other equally suitable material or combinations of materials that are readily known to those of ordinary skill in the art as a result of routine investigation and testing.

As further illustrated by FIG. 5, in use, the first responder may slide the skin protecting tourniquet apparatus 10 around the limb A. They may then insert the tail 12 into the mouth 22 in the head 16, and pull the tail 12 through until the locking teeth 14 begin to engage to substantially lock the length of the skin protecting tourniquet apparatus 10. The first responder may then adjust and/or tighten the skin protecting tourniquet apparatus 10 until they achieve the proper location and pressure around the limb A. While doing so, the skin protecting tourniquet apparatus 10 configuration may substantially avoid pinching the patient's skin. Thereafter, the first responder may write the time and date they applied the skin protecting tourniquet apparatus 10. The length of an individual skin protecting tourniquet apparatus 10 may allow it to be used on arms as well as legs yet still couple to and/or fit onto personal equipment (e.g., a MOLLE/PALS compatible flak jacket or vest).

Depending on the application and/or size of the patient's arm leg, or overall build, multiple skin protecting tourniquet apparatuses 10 may link together to increase the effective length of the skin protecting tourniquet apparatus 10. For example, the tail 12 of a first skin protecting tourniquet apparatus 10 may insert into the mouth 22 of a second skin protecting tourniquet apparatus 10 while the tail 12 of the second skin protecting tourniquet apparatus 10 may insert into the mouth 22 of the first skin protecting tourniquet apparatus 10, thereby approximately doubling the length of the skin protecting tourniquet apparatus 10 of an embodiment.

Those skilled in the art will recognize that numerous modifications and changes may be made to the preferred embodiment without departing from the scope of the claimed invention. It will, of course, be understood that modifications of the invention, in its various aspects, will be apparent to those skilled in the art, some being apparent only after study, others being matters of routine mechanical, chemical and electronic design. No single feature, function or property of the preferred embodiment is essential. Other embodiments are possible, their specific designs depending upon the particular application. As such, the scope of the invention should not be d by the particular embodiments herein described but should be defined only by the appended claims and equivalents thereof.

I claim:

1. A tourniquet comprising:
a tail including a first end and a second end
a tip coupled to the first end of the tail;
a hood coupled to the second end of the tail;
a neck coupled to the hood opposite the tail;
a head coupled to the neck, the head further comprising a mouth to receive the tip and at least a portion of the tail;
wherein an intersection of the neck and the hood define an angle.

2. The tourniquet of claim 1, the tail further comprising one or more locking teeth, the mouth to engage the one or more locking teeth when the tail is inserted in the mouth.

3. The tourniquet of claim 2, the mouth to substantially unidirectionally engage the one or more locking teeth when the tail is inserted in the mouth to substantially prevent the e oval of the tail from the mouth.

4. The tourniquet of claim 1, the angle further comprising an angle approximately between 20 and 40 degrees.

5. The tourniquet of claim 1, the angle further comprising an angle of approximately 30 degrees.

6. The tourniquet of claim 4, the angle to substantially prevent a user's skin from being pinched when the tourniquet is applied to the user.

7. A tourniquet comprising:
a tail including a plurality of locking teeth;
a neck coupled to the tail; and
a head coupled to the neck, the head including a locking mechanism to detachably engage the locking teeth, the neck and the tail to define an angle therebetween.

8. The tourniquet of claim 7 wherein the angle defined by the neck and the tail further comprises and angle approximately between 20 degrees and 40 degrees.

9. The tourniquet of claim 7 wherein the angle defined by the neck and the tail further comprises an angle of approximately 30 degrees.

10. The tourniquet of claim 7, the angle defined by the neck and the tail to substantially prevent pinching the skin over which the tourniquet is applied.

11. The tourniquet of claim 7, the angle defined by the neck and the tail to substantially securely couple the tourniquet to a pouch attachment ladder system (PALS) webbing when the tourniquet is inserted therein.

12. The tourniquet of claim 7 further comprising a hood coupled to the neck adjacent the head on which data may be recorded and/or written.
